(19) United States
(12) Patent Application Publication
(10) Pub. No.: US 2013/0304151 A1
(43) Pub. Date: Nov. 14, 2013

(54) STIMULATION METHOD FOR TREATMENT OF MEDICAL CONDITIONS

(71) Applicant: Autonomic Technologies, Inc., Redwood City, CA (US)

(72) Inventors: Amy M. Goodman, San Francisco, CA (US); Anthony V. Caparso, San Francisco, CA (US); Benjamin D. Pless, Atherton, CA (US)

(21) Appl. No.: 13/917,994

(22) Filed: Jun. 14, 2013

Related U.S. Application Data
(63) Continuation-in-part of application No. 13/476,224, filed on May 21, 2012.
Application No. 13/476,224 is a continuation-in-part of application No. 13/470,480, filed on May 14, 2012.
(60) Provisional application No. 61/659,702, filed on Jun. 14, 2012.

(51) Int. Cl. A61N 1/36

(52) U.S. Cl. CPC A61N 1/36
USPC 607/40; 607/2; 607/45; 607/46; 607/48

(57) ABSTRACT

One aspect of the present disclosure relates to a method for modulating, suppressing or preventing a medical condition in a subject. One step of the method can include positioning at least one electrode on or proximate to at least one of a vidian nerve (VN), a greater petrosal nerve (GPN), a deep petrosal nerve (DPN), or a branch thereof, of the subject. Next, the at least one electrode can be activated to apply an electrical signal to at least one of the VN, the GPN, the DPN, or the branch thereof.

### RELATED APPLICATIONS

The present application is a continuation-in-part of U.S. patent application Ser. Nos. 13/476,224, filed May 21, 2012, and 13/470,480, filed May 14, 2012, and also claims the benefit of U.S. Provisional Patent Application Ser. No. 61/659,702, filed Jun. 14, 2012. Each of the aforementioned applications is hereby incorporated by reference in its entirety.

### TECHNICAL FIELD

The present disclosure relates generally to neuromodulatory methods, and more particularly to methods for treating medical conditions by stimulation of a vidian nerve, a greater petrosal nerve, a deep petrosal nerve, and/or a branch thereof.

### BACKGROUND

The autonomic nervous system regulates certain body processes, such as blood pressure and the rate of breathing. This system works automatically (autonomously), without a person's conscious effort. The autonomic nervous system has two main divisions: the sympathetic and the parasympathetic. After the autonomic nervous system receives information about the body and external environment, it responds by stimulating body processes, usually through the sympathetic division, or inhibiting them, usually through the parasympathetic division. Disorders of the autonomic nervous system can affect any body part or process. Autonomic disorders may result from other disorders that damage autonomic nerves (such as diabetes), or they may occur on their own. Autonomic disorders may be reversible or progressive.

### SUMMARY

The present disclosure relates generally to neuromodulatory methods, and more particularly to methods for treating medical conditions by stimulation of a vidian nerve (VN), a greater petrosal nerve (GPN), a deep petrosal nerve (DPN), and/or a branch thereof.

One aspect of the present disclosure relates to a method for suppressing or preventing a medical condition in a subject. One step of the method can include positioning at least one electrode on or proximate to at least one of a VN, a GPN, a DPN, or a branch thereof, of the subject. Next, the at least one electrode can be activated to apply an electrical signal to at least one of the VN, the GPN, the DPN, or the branch thereof.

Another aspect of the present disclosure relates to a method for suppressing or preventing a medical condition in a subject. One step of the method can include positioning at least one electrode on or proximate to at least one of a VN, a GPN, a DPN, or a branch thereof, of the subject. Next, the at least one electrode can be activated to apply an electrical signal to at least one of the VN, the GPN, the DPN, or the branch thereof. The medical condition is selected from the group consisting of pain, movement disorders, epilepsy, cerebrovascular diseases, autoimmune diseases, sleep disorders, autonomic disorders, urinary bladder disorders, abnormal metabolic states, disorders of the muscular system, cardiovascular disorders, pulmonary disorders, inflammatory disorders, and neuropsychiatric disorders.

Another aspect of the present disclosure relates to a method for suppressing or preventing pain in a subject. One step of the method can include positioning at least one electrode on or proximate to at least one of a VN, a GPN, a DPN, or a branch thereof, of the subject. Next, the at least one electrode can be activated to apply an electrical signal to at least one of the VN, the GPN, the DPN, or the branch thereof.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration of a lateral view of a human skull showing the sphenopalatine ganglion lying within the sphenopalatine fossa, the vidian nerve, the greater petrosal nerve, and the deep petrosal nerve; and

FIG. 2 is a schematic illustration showing the geniculate ganglion and its associated branches.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the term “in communication” can refer to at least a portion of an electrode being adjacent, in the general vicinity, in close proximity, or directly next to and/or directly on a target nerve or nerve structure, such as a vidian nerve (VN) (also called “the nerve of the pterygoid canal”), a greater petrosal nerve (GPN), a deep petrosal nerve (DPN), or a branch thereof (e.g., a nasopalatine nerve, a greater palatine nerve, a lesser palatine nerve, a superior posterior alveolar nerve, or a lesser petrosal nerve). In some instances, the term can mean that at least a portion of an electrode is “in communication” with a target nerve or nerve structure if application of a therapy signal (e.g., an electrical signal) thereto results in a modulation of neuronal activity to elicit a desired response, such as modulation of a nerve signal (e.g., an action potential or electrical impulse) generated in, or transmitted through, the target nerve or nerve structure.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, etc.

As used herein, the terms “modulate” or “modulating” with reference to activity of a target nerve or nerve structure can refer to causing a change in neuronal activity, chemistry and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of neuronal activity.

As used herein, the terms “substantially blocked” or “substantially block” when used with reference to activity of a target nerve or nerve structure can refer to a complete (e.g., 100%) or partial inhibition (e.g., less than 100%, such as about 90%, about 80%, about 70%, about 60%, or less than about 50%) of nerve conduction therethrough. For example, the terms “block”, “blocking”, and “blockade” can refer to the disruption, modulation, and/or inhibition of nerve impulse transmissions through a target nerve or nerve structure.

As used herein, the term “activity” when used with reference to a target nerve or nerve structure can, in some instances, refer to the ability of a nerve, neuron, or fiber to conduct, propagate, and/or generate an action potential. In other instances, the term can refer to the frequency at which a nerve or neuron is conducting, propagating, and/or generating one or more action potentials at a given moment in time. In further instances, the term can refer to the frequency at which a nerve or neuron is conducting, propagating, and/or generating one or more action potentials over a given period of time (e.g., seconds, minutes, hours, days, etc.).

As used herein, the term “electrical communication” can refer to the ability of an electric field generated by an electrode or electrode array to be transferred, or to have a neuromodulatory effect, within and/or on a nerve, neuron, or fiber of a target nerve or nerve structure.

As used herein, the terms “prevent” or “preventing” when used with reference to a medical condition can refer to stopping a medical condition from occurring, or taking advance measures against the possibility or probability that a medical condition will happen or occur. In some instances, the terms can refer to an action or actions taken to decrease the chance that a subject will contract, develop, or suffer from a medical condition.

As used herein, the terms “suppress” or “suppressing” when used with reference to a medical condition can refer to refer to any quantitatively or qualitatively measurable or observable reduction or attenuation in a medical condition (e.g., a sign or symptom associated with the medical condition).

As used herein, the term “medical condition mediated by autonomic or neurological dysfunction” can refer to any condition, state, or disease that is characterized, at least in part, by a disruption in nerve signals (e.g., action potentials or electrical impulses) passing through or associated with the autonomic nervous system (ANS). Such medical conditions can result from, be caused by (e.g., directly or indirectly), or otherwise be associated with autonomic or neurological dysfunction. Non-limiting examples of medical conditions mediated by autonomic or neurological dysfunction are provided below.

As used herein, the terms “treat” or “treating” can refer to therapeutically regulating, preventing, improving, alleviating the symptoms of, and/or reducing the effects of a medical condition. As such, treatment also includes situations where a medical condition, or at least symptoms associated therewith, is completely inhibited, e.g., prevented from happening or stopped (e.g., terminated) such that the subject no longer suffers from the medical condition, or at least the symptom(s) that characterize the medical condition.

### Overview

A brief discussion of the pertinent neurophysiology is provided to assist the reader with understanding certain aspects of the present disclosure.

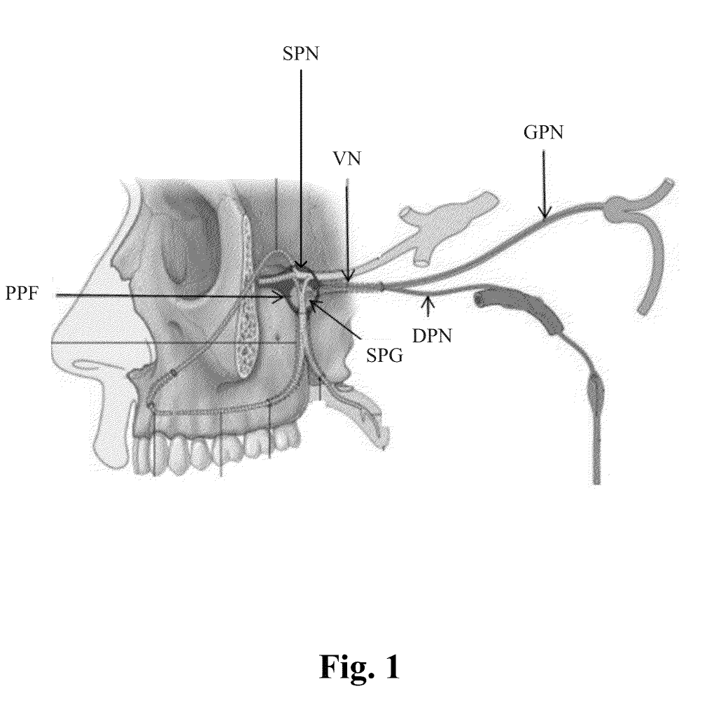

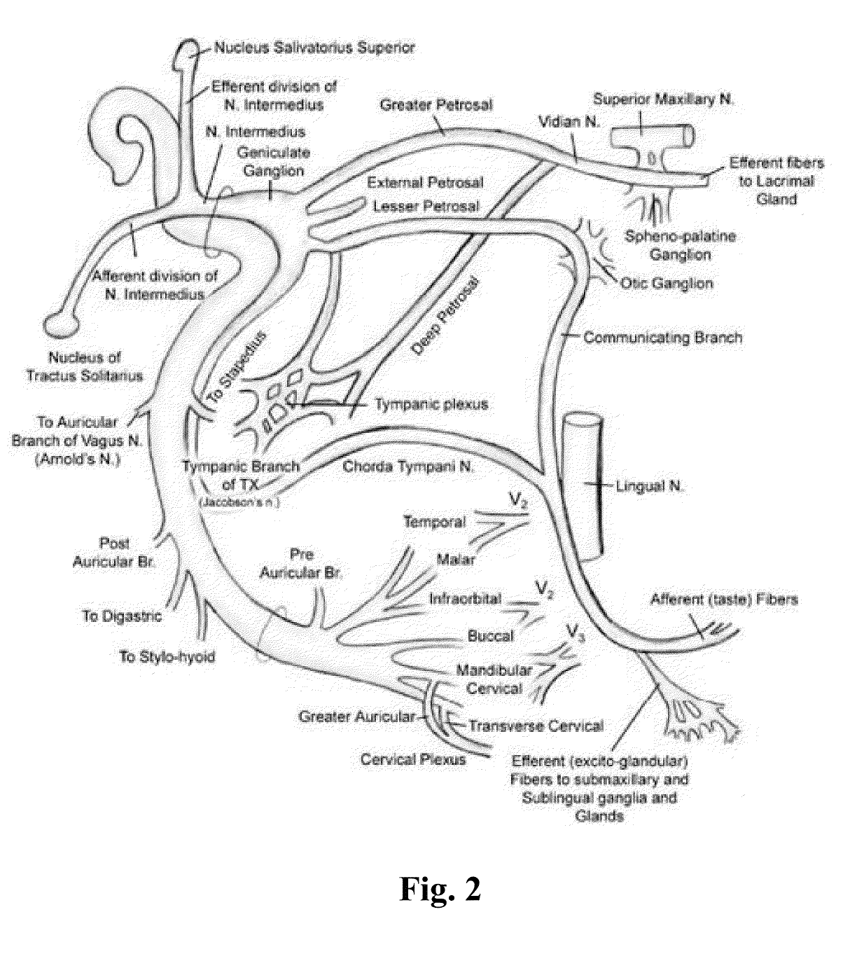

The SPG (FIGS. 1-2), also called the pterygopalatine ganglion, is located within the pterygopalatine fossa (PPF). The PPF is bounded anteriorly by the maxilla, posteriorly by the medial plate of the pterygoid process and greater wing of the sphenoid process, medially by the palatine bone, and superiorly by the body of the sphenoid process. Its lateral border is the pterygomaxillary fissure, which opens to the infratemporal fossa.

The SPG is a large, extra-cranial parasympathetic ganglion. The SPG is a complex neural ganglion with multiple connections, including autonomic, sensory and motor. The maxillary branch of the trigeminal nerve and the nerve of the pterygoid canal, also known as the VN sends neural projections to the SPG. The fine branches from the maxillary nerve—known as the pterygopalatine nerves or SPN—form the sensory component of the SPG. The SPN pass through the SPG and do not synapse. The GPN (discussed below) carries the preganglionic parasympathetic axons from the superior salivary nucleus to the SPG. These fibers synapse onto the postganglionic neurons within the SPG. The DPN (discussed below) connects the superior cervical sympathetic ganglion to the SPG and carries postganglionic sympathetic axons that again pass through the SPG without any synapses. The DPN and the GPN carry sympathetic and parasympathetic fibers, respectively. The greater and lesser palatine nerves are branches of the SPG that carry both general sensory and parasympathetic fibers.

The DPN and the GPN join together just before entering the pterygoid canal to form the VN. The DPN is given off from the carotid plexus and runs through the carotid canal lateral to the internal carotid artery. It contains postganglionic sympathetic fibers with cell bodies located in the superior cervical ganglion. It then enters the cartilaginous substance, which fills the foramen lacerum, and joins with the greater superficial petrosal nerve to form the VN. The GPN then passes through the SPG without synapsing, and joins the postganglionic parasympathetic fibers in supplying the lacrimal gland, the nasal mucosa, and the oral mucosa. The GPN is given off from the geniculate ganglion of the facial nerve. It passes through the hiatus of the facial canal, enters the cranial cavity, and runs forward beneath the dura mater in a groove on the anterior surface of the petrous portion of the temporal bone. The GPN enters the cartilaginous substance, which fills the foramen lacerum, and then joins with the DPN to form the VN. The lesser petrosal nerve carries parasympathetic (secretory) fibers from both the tympanic plexus and the nervus intermedius to the parotid gland. The lesser petrosal nerve originates at the geniculate ganglion and passes forwards through its own canal back into the middle cranial fossa.

The VN is housed within the Vidian canal, which is posterior to the SPG. The VN connects to the SPG and contains parasympathetic fibers, which synapse in the SPG, sensory fibers that provide sensation to part of the nasal septum, and also sympathetic fibers. The SPN are sensory nerves that connect the SPG to the maxillary nerve. The SPN traverse through the SPG without synapsing and proceed to provide sensation to the palate. The SPN suspend the SPG in the PPF.

The present disclosure relates generally to neuromodulatory methods, and more particularly to methods for treating medical conditions in a subject. Without wishing to be bound by theory, it is believed that abnormal regulation of pain or autonomic pathways, which may be a feature of the medical conditions disclosed herein, can cause excitation, loss of inhibition, suppression, or loss of excitation of these pathways. Thus, in some instances, the present disclosure provides methods for applying one or more therapy signals to a target nerve or nerve structure, such as a VN, a GPN, a DPN, and/or a branch thereof (e.g., a nasopalatine nerve, a greater palatine nerve, a lesser palatine nerve, a superior posterior alveolar nerve, or a lesser petrosal nerve) to modulate the transmission of nerve signals and stimulate or block the autonomic pathways passing through the target nerve or nerve structure to modulate, reduce or eliminate one or more symptoms or signs associated with the medical condition. In other instances, it is similarly believed that application of one or more therapy signals to a target nerve or nerve structure (e.g., a VN, a GPN, a DPN and/or a branch thereof) can modulate the transmission of nerve signals other than pain responsible for provoking or aggravating other undesirable sensations or conditions.

### Methods

One aspect of the present disclosure can include a method for modulating, suppressing, preventing, or treating a medical condition (e.g., mediated by autonomic or neurological dysfunction) in a subject. Methods of the present disclosure can generally include the steps of: positioning at least one electrode on or proximate to a target nerve or nerve structure (e.g., a VN, a GPN, a DPN and/or a branch thereof) of the subject; and activating the at least one electrode to apply a therapy signal (e.g., an electrical signal) to the target nerve or nerve structure. In some instances, the methods of the present disclosure can act to suppress or prevent a medical condition by disrupting nerve signals passing through the ANS as the signals traverse or are generated in the target nerve or nerve structure. To treat a medical condition mediated by dysfunction of the parasympathetic nervous system, for example, the GPN and/or a branch thereof may be modulated according to the present disclosure. Alternatively, to treat a medical condition mediated by dysfunction of the sympathetic nervous system, the DPN and/or a branch thereof may be modulated according to the present disclosure.

In some instances, medical conditions (e.g., mediated or caused by autonomic or neurological dysfunction) that can be modulated, suppressed, prevented, or treated by the present disclosure can include pain, movement disorders, epilepsy, cerebrovascular diseases, autoimmune diseases, sleep disorders, autonomic disorders, urinary bladder disorders, abnormal metabolic states, disorders of the muscular system, cardiovascular disorders, pulmonary disorders, inflammatory disorders, and neuropsychiatric disorders.

Pain treatable by the present disclosure can include migraine headaches, including migraine headaches with aura, migraine headaches without aura, menstrual migraines, migraine variants, atypical migraines, complicated migraines, hemiplegic migraines, transformed migraines, and chronic daily migraines, episodic tension headaches, chronic tension headaches, analgesic rebound headaches, episodic cluster headaches, chronic cluster headaches, cluster variants, chronic paroxysmal hemicranias, hemicrania continua, post-traumatic headache, post-traumatic neck pain, post-herpetic neuralgia involving the head or face, pain from spine fracture secondary to osteoporosis, arthritis pain in the spine, headache related to cerebrovascular disease and stroke, headache due to vascular disorder, reflex sympathetic dystrophy, cervicalgia (which may be due to various causes, including, but not limited to, muscular, discogenic, or degenerative, including arthritic, posturally related, or metastatic), glossodynia, carotidynia; cricoidynia, otalgia due to middle ear lesion, gastric pain, sciatica, maxillary neuralgia, laryngeal pain, myalgia of neck muscles, trigeminal neuralgia (sometimes also termed tic douloureux), post-lumbar puncture headache, low cerebro-spinal fluid pressure headache, temporomandibular joint disorder, atypical facial pain, ciliary neuralgia, paratrigeminal neuralgia (sometimes also termed Raeder's syndrome), petrosal neuralgia, Eagle's syndrome, idiopathic intracranial hypertension, orofacial pain, myofascial pain syndrome involving the head, neck, and shoulder, chronic migraneous neuralgia, cervical headache, paratrigeminal paralysis, sphenopalatine ganglion neuralgia (sometimes also termed lower-half headache, lower facial neuralgia syndrome, Sluder's neuralgia, and Sluder's syndrome), carotidynia, Vidian neuralgia, causalgia, atypical odontalgia, cluster tic syndrome, geniculate neuralgia, glossopharyngeal neuralgia, occipital neuralgia, temporal arteritis, or a combination thereof.

Movement disorders treatable by the present disclosure can include Parkinson's disease, cerebropalsy, dystonia, essential tremor, hemifacial spasms, or a combination thereof.

Epilepsy treatable by the present disclosure can include generalized or partial epilepsy.

Cerebrovascular diseases treatable by the present disclosure can include aneurysms, strokes, cerebral hemorrhage, or a combination thereof.

Autoimmune diseases treatable by the present disclosure can include multiple sclerosis.

Sleep disorders treatable by the present disclosure can sleep apnea, parasomnias, or a combination thereof.

Autonomic disorders treatable by the present disclosure can include gastrointestinal motility disorders, such as nausea, vomiting, diarrhea, chronic hiccups, gastroesphageal reflux disease, and hypersecretion of gastric acid, autonomic insufficiency, excessive epiphoresis and excessive rhinorrhea, as well as cardiovascular disorders, such as cardiac dysrythmias and arrythmias, hypertension, carotid sinus disease, Holmes-adie syndrome, orthostatic hypotension, striatonigral degeneration, vasovagal syncope, Lyme disease, autonomic instability, or a combination thereof.

Neurological disorders treatable by the present disclosure can include hemifacial spasm, Melkersson-Rosenthal Syndrome, Parry-Romberg syndrome, or a combination thereof.

Urinary bladder disorders treatable by the present disclosure include spastic or flaccid bladder.

Abnormal metabolic states treatable by the present disclosure can include hyperthyroidism or hypothyroidism.

Disorders of the muscular system treatable by the present disclosure can include muscular dystrophy and spasms of the upper respiratory tract and face.

Neuropsychiatric disorders treatable by the present disclosure can include depression, schizophrenia, bipolar disorder, obsessive-compulsive disorder, or a combination thereof.

Pulmonary disorders treatable by the present disclosure can include both infection- and non-infection-induced disease and dysfunction of the respiratory system. Non-limiting examples of pulmonary conditions include genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, brochiectasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

Cardiovascular disorders treatable by the present disclosure can encompass the entire vascular system of the body (including the heart itself), and include ischemic heart disease, angina pectoris, coronary heart disease, stroke, transient ischemic attacks, hypertensive disease, aortic aneurysm, peripheral arterial disease, retinal arterial disease and heart failure.

Neurovascular disorders treatable the present disclosure can include any disorder characterized by one or more disturbances in the normal functioning of at least one component of the cerebral vascular or cerebral nervous system in a subject including, but not limited to, peripheral vascular disease and CPRS.

In one example, a medical condition (e.g., mediated or caused by autonomic or neurological dysfunction) that can be suppressed, prevented, or treated by the present disclosure can include pain. In some instances, and without wishing to be bound by theory, it is believed that the abnormal regulation of pain pathways can cause excitation or a loss of inhibition of those pathways, resulting in an increased perception of pain. Thus, applying one or more therapy signals (e.g., an electrical signal) to a target nerve or nerve structure (e.g., a VN, a GPN, a DPN and/or a branch thereof) can substantially block the transmission of pain signals (e.g., aberrant or abnormal sensory signals) and stimulate inhibitory feedback of the pain pathways passing therethrough to reduce or eliminate pain experienced by the subject.

In another aspect, the at least one electrode can include any mono-polar, bipolar, or mutli-polar electrode configured to deliver an electrical signal to a target nerve or nerve structure (e.g., a VN, a GPN, a DPN and/or a branch thereof). In some instance, the at least one electrode can be securely disposed on or within a housing or casing (e.g., made of silicon, metal or plastic). In other instances, the at least one electrode can be securely disposed on a percutaneous lead. Alternatively, the electrode can be configured as a cuff-type electrode. In further instances, the at least one electrode can comprise one component of a neurostimulator. In such instances, the neurostimulator can comprise any active implantable medical device configured to deliver electrical stimulation, alone or in combination with other types of stimulation, to a target nerve or nerve structure (e.g., a VN, a GPN, a DPN and/or a branch thereof) of a subject. The neurostimulator can further include any active implantable medical device configured for implantation for a relatively short period of time (e.g., to address acute medical conditions) or a relatively long period of time (e.g., to address chronic medical conditions). Additionally, the neurostimulator can include one or more elements used to record or monitor a physiological response of a subject's tissue (e.g., a delivered therapy), as well as one or more other components that interface with the subject's tissue (e.g., therapeutic agent delivery mechanisms, sensors, etc.). The neurostimulator can further include, or at least be in electrical communication with, a power source that provides the energy source for electrical stimulation.

One or a combination of surgical methods may be used to implant the at least one electrode on or adjacent a target nerve or nerve structure (e.g., a VN, a GPN, a DPN and/or a branch thereof) such that the at least one electrode is in electrical communication with the target nerve or nerve structure. In some instances, a percutaneous technique can be used to implant the at least one electrode. Examples of percutaneous techniques that may be employed are disclosed in U.S. Pat. No. 6,526,318 (hereinafter, “the '318 patent”), as well as U.S. patent application Ser. Nos. 13/476,224 (hereinafter, “the “224 application”) and 13/470,480 (hereinafter, “the '480 application”). Because the VN, the GPN, and the DPN are in relatively close proximity to one another within a very small area, the same technique can be applied to achieve placement of at least one electrode on or adjacent to any of the three structures. It should also be understood that, because the region in which the VN, the GPN, and the DPN all join together is very small, stimulation of the VN, GPN, DPN and/or a branch thereof, even when an electrode is placed optimally, may also stimulate two or all of the other structures.

It will also be understood that surgical methods other than percutaneous approaches may be used to implant an electrode on or proximate to a target nerve or nerve structure. In one example, at least one electrode can be positioned on the skin of a subject adjacent a target nerve or nerve structure (e.g., a VN, a GPN, a DPN and/or a branch thereof) so that an electrical signal can be transcutaneously delivered to the target nerve or nerve structure. In another example, an intravascular approach can be used so that at least one electrode is positioned adjacent (e.g., directly adjacent) a target nerve or nerve structure. An electrical signal can then be delivered to the electrode so that electrical energy is transvascularly delivered to the target nerve or nerve structure.

In some instances, the at least one electrode can be implanted in the subject without penetrating the cranium of the subject.

In other instance, the at least one electrode can be implanted in the subject without penetrating the nasal cavity or the palate of the subject.

In another aspect, a therapy signal can be applied to the target nerve or nerve structure (e.g., a VN, a GPN, a DPN and/or a branch thereof) to modulate activity associated with the target nerve or nerve structure and thereby prevent or suppress the medical condition. Neuromodulation of the VN, for example, can be done directly or indirectly by affecting postganglionic neurons located within the VN and/or their corresponding axons, or the preganglionic axons in the VN that synapse with the SPG, respectively. Examples of therapy signals that may be applied to a target nerve or nerve structure can include electrical energy, chemical agents, mechanical force, thermal energy, and combinations thereof.

In some instances, the therapy signal can be an electrical signal. Electrical stimulation may be delivered in any of several forms, such as biphasic charge-balanced pulses having a frequency of about 1-1000 Hz (e.g., 5-200 Hz), a pulse-width of about 0.04-2 ms, a current of about 0.05-100 mA (e.g., 0.1-5 mA), and a voltage of about 1-10 V. In addition, electrical modulation can be controllable such that either anodic or cathodic stimulation may be applied. Stimulation may be delivered continuously, intermittently, as a burst in response to a control signal, or as a burst in response to a sensed parameters, such as increased SPG neural activity. The electrical parameters may also be adjusted automatically based on a control signal, based on sensed parameters, or by selection by the subject.

In some instances, electrical energy can be applied to a target nerve or nerve structure (e.g., a VN, a GPN, a DPN and/or a branch thereof) for a time and in an amount insufficient to cause a lesion on the target nerve or nerve structure.

In another aspect, a device that includes at least one electrode may be utilized which, instead of or in addition to delivering electrical stimulation to the target nerve or nerve structure, delivers a medication solution or analgesic to the target nerve or nerve structure. For example, a device having at least one electrode and a small port at its tip, which is connected to a reservoir or medication pump containing a medication solution or an analgesic (e.g., an anesthetic solution) may be used. The medication/analgesic delivery device may be implanted using the same procedure as used for the electrical stimulation electrode. If desired by the subject or physician, the reservoir or medication pump may also be implanted in the subject's body (e.g., similar or identical to an implantable pulse generator). In some instances, the device can be controllable such that the amount of medication solution or analgesic applied, the rate at which medication solution or analgesic is applied, and the time period over which the medication solution or analgesic is applied is adjustable.

It should be understood that delivery of a medication solution or analgesic from a device may be used alone or in conjunction with the electrical stimulation method described above. For example, a device may be used that is capable of either producing an electrical signal or delivering a medication solution or analgesic. As another example, an electrostimulatory approach could be applied to a target nerve or nerve structure (e.g., a VN, a GPN, a DPN and/or a branch thereof) of one side of a subject's face, while the method utilizing delivery of a medication solution or analgesic could be applied to the same or different target nerve or nerve structure on the other side of the subject's face.

Advantageously, once the at least one electrode is placed into communication with the target nerve or nerve structure, application of one or more therapy signals (e.g., electrical signals) can be adjusted to the subject's individual needs (e.g., by the subject or via a closed-loop system) without requiring further surgical intervention.

In one example of the present disclosure, a neurostimulator (not shown) can be implanted in or about the PPF to deliver an electrical signal to a VN and/or a branch thereof. The neurostimulator can be configured identically or similarly as the neurostimulator disclosed in the '224 application. For instance, the neurostimulator can include a pulse generator, an integral lead system, and an integral fixation plate. The neurostimulator can be delivered to the PPF in an identical or similar fashion as disclosed in the '480 application. Briefly, for example, a gingival-buccal surgical approach can be used whereby a trans-oral incision is first created. An introducer (not shown) is then inserted into the incision and advanced posteriorly, superiorly and medially toward the PPF. The introducer is carefully advanced so as to maintain contact with the posterior maxilla.

Once a distal end of the introducer is placed within the PPF, the neurostimulator can be advanced within a predefined groove of the introducer into the PPF. The neurostimulator is surgically placed such that the integral lead (with at least one stimulation electrode (not shown)) located within the PPF directly on or adjacent to the VN and/or a branch thereof. The integral fixation plate of the neurostimulator is securely anchored to the zygomatic process of the maxilla. Following fixation of the neurostimulator, the neurostimulator can be activated so that the stimulation electrode delivers an electrical signal to the VN and/or a branch thereof to modulate (e.g., substantially block) nerve signal transmission therethrough.

In another example of the present disclosure, an intravascular approach may be used to deliver one or more electrical signals to the DPN and/or a branch thereof. In some instances, an appropriately-sized intravascular device, such as one identically or similarly configured as the devices disclosed in U.S. patent application Ser. No. 11/641,331 may be used. In such instances, the intravascular device may be advanced through the vasculature (e.g., an artery or vein) of a subject to a location adjacent (e.g., directly adjacent) or proximate to the DPN and/or a branch thereof such that delivery of an electrical signal to the intravascular device is effective to modulate autonomic activity associated therewith (e.g., modulating sympathetic activity). In one example, an intravascular device can be positioned at a location in the internal carotid artery adjacent (e.g., directly adjacent) or proximate to the DPN and/or a branch thereof such that delivery of an electrical signal to the intravascular device is effective to modulate sympathetic activity in the subject. In another example, an intravascular device can be positioned at a location in the internal jugular vein adjacent (e.g., directly adjacent) or proximate to the DPN and/or a branch thereof such that delivery of an electrical signal to the intravascular device is effective to modulate sympathetic activity in the subject. It will be appreciated that electrical signals can be delivered to the intravascular device either directly (e.g., via a lead) or wirelessly. Alternatively, the intravascular device may be self-powered by, for example, a battery that may be remotely or inductively charged as needed.

In another example of the present disclosure, one or a combination of percutaneous techniques may be used to target the GPN and/or a branch thereof such that delivery of an electrical signal to the at least one electrode is effective to modulate autonomic activity associated therewith (e.g., parasympathetic activity). In one approach, an electrode may be placed on or proximate to the GPN and/or a branch thereof via the paryngotympanic tube, which may be accessed via the nasopharynx of a subject. In another approach, at least one electrode may be placed on or proximate to the GPN and/or a branch thereof via the backside of the inner ear. Alternatively, an electrode may be placed on a portion of the wall of Meckel's cave, which may be accessed via the foramen ovale such that delivery of an electrical signal to the at least one electrode is effective to modulate parasympathetic activity in the subject.

From the above description of the present disclosure, those skilled in the art will perceive improvements, changes and modifications. For example, it will be appreciated that the methods of the present disclosure can be performed to apply modulate activity of a target nerve or nerve structure (e.g., a VN, a GPN, a DPN and/or a branch thereof) on either or both sides of a subject's head. Such improvements, changes, and modifications are within the skill of those in the art and are intended to be covered by the appended claims. All patents, patent applications, and publication cited herein are incorporated by reference in their entirety.

The following is claimed:

1. A method for modulating, suppressing or preventing a medical condition in a subject, the method comprising the steps of:
positioning at least one electrode on or proximate to at least one of a vidian nerve (VN), a greater petrosal nerve (GPN), a deep petrosal nerve (DPN), or a branch thereof, of the subject; and
activating the at least one electrode to apply an electrical signal to at least one of the VN, the GPN, the DPN, or the branch thereof.

2. The method of claim 1, wherein the medical condition is mediated by autonomic or neurological dysfunction.

3. The method of claim 2, further comprising the step of disrupting nerve signal generation in, or transmission through, at least one of the VN, the GPN, the DPN, or the branch thereof.

4. The method of claim 1, wherein the positioning step further comprises advancing the at least one electrode, without penetrating the cranium, into the pterygopalatine fossa so that the at least one electrode is positioned on or proximate to at least one of the VN or the branch thereof.

5. The method of claim 1, wherein the at least one electrode is advanced without penetrating the nasal cavity or the palate.

6. The method of claim 1, wherein the activating step generates heat insufficient to cause a lesion on at least one of the VN, the GPN, the DPN, or the branch thereof.

7. The method of claim 1, further including the step of adjusting the electrical signal without requiring an invasive procedure on the subject.

8. The method of claim 1, wherein the at least one electrode is positioned proximate to the GPN, the DPN, or a branch thereof, via an intravascular route.

9. A method for suppressing or preventing a medical condition in a subject, the method comprising the steps of:
positioning at least one electrode on or proximate to at least one of a VN, a GPN, a DPN, or a branch thereof, of the subject; and
activating the at least one electrode to apply an electrical signal to at least one of the VN, the GPN, the DPN, or the branch thereof;
wherein the medical condition is selected from the group consisting of pain, movement disorders, epilepsy, cerebrovascular diseases, autoimmune diseases, sleep disorders, autonomic disorders, urinary bladder disorders, abnormal metabolic states, disorders of the muscular system, cardiovascular disorders, pulmonary disorders, inflammatory disorders, and neuropsychiatric disorders.

10. The method of claim 9, wherein the positioning step further comprises advancing the at least one electrode, without penetrating the cranium, into the pterygopalatine fossa so that the at least one electrode is positioned on or proximate to at least one of the VN or the branch thereof.

11. The method of claim 9, wherein the at least one electrode is advanced without penetrating the nasal cavity or the palate.

12. The method of claim 9, wherein the activating step generates heat insufficient to cause a lesion on at least one of the VN, the GPN, the DPN, or the branch thereof.

13. The method of claim 9, wherein the at least one electrode is positioned proximate to the GPN, the DPN, or a branch thereof, via an intravascular route.

14. A method for modulating, suppressing or preventing pain in a subject, the method comprising the steps of:
positioning at least one electrode on or proximate to at least one of a VN, a GPN, a DPN, or a branch thereof, of the subject; and
activating the at least one electrode to apply an electrical signal to at least one of the VN, the GPN, the DPN, or the branch thereof.

15. The method of claim 14, wherein the pain is mediated by autonomic dysfunction or neurological dysfunction.

16. The method of claim 15, further comprising the step of disrupting pain signal generation in, or transmission through, at least one of the sphenopalatine ganglion, the VN or the branch thereof.

17. The method of claim 14, wherein the positioning step further comprises advancing the at least one electrode, without penetrating the cranium, into the pterygopalatine fossa so that the at least one electrode is positioned on or proximate to the VN or the branch thereof.

18. The method of claim 14, wherein the at least one electrode is advanced without penetrating the nasal cavity or the palate.

19. The method of claim 14, wherein the activating step generates heat insufficient to cause a lesion on at least one of the VN, the GPN, the DPN, or the branch thereof.

20. The method of claim 14, wherein the at least one electrode is positioned proximate to the GPN, the DPN, or a branch thereof, via an intravascular route.
